(19) United States
(12) Patent Application Publication
(10) Pub. No.: US 2010/0256379 A1
(43) Pub. Date: Oct. 7, 2010

(54) ANTI-VIRAL AGENT CONTAINING HETEROCYCLIC AROMATIC COMPOUND AS ACTIVE INGREDIENT

(75) Inventors: Masanori Baba, Kagoshima (JP); Yuichi Hashimoto, Bunkyo-ku (JP); Hiroshi Aoyama, Bunkyo-ku (JP); Kumiko Sako, Bunkyo-ku (JP); Shinichi Sato, Bunkyo-ku (JP); Katsuyuki Nagai, Minato-ku (JP)

Correspondence Address: FOLEY AND LARDNER LLP;SUITE 500, 3000 K STREET NW, WASHINGTON, DC, 20007, US

(73) Assignees: Kagoshima University; Oncolys Biopharma, Inc.

(21) Appl. No.: 12/739,795

(22) PCT Filed: Oct. 22, 2008
(86) PCT No.: PCT/JP2008/069122

§ 371 Date: Apr. 26, 2010

(30) Foreign Application Priority Data
Oct. 26, 2007 (JP) 2007-279648
Feb. 29, 2008 (JP) 2008-050771

(51) Int. Cl. C07D 471/04

(52) U.S. Cl. 546/85

(57) ABSTRACT

The present invention relates to an anti-viral agent comprising a compound represented by the following formula (I):

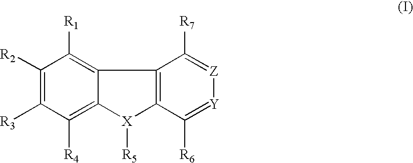

(wherein X represents CH, a nitrogen atom, an oxygen atom, or a sulfur atom;
Y and Z are the same or different and each represents a nitrogen atom or C—R₈, and at least one of them represents a nitrogen atom;
R₁ to R₈ are the same or different and each represents a hydrogen atom, a linear C₁₋₁₀-hydrocarbon group, a hydroxy group, or a substituted or unsubstituted benzyl group; and,
when X represents an oxygen atom or a sulfur atom, R₅ is absent), or the following formula (II):

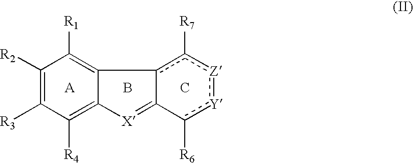

(wherein X′ represents CH or a nitrogen atom;
Y′ and Z′ are the same or different and each represents a nitrogen atom or N—R₉, or C—R₈ and at least one of them represents a nitrogen atom or N—R₉;
R₁ to R₄ and R₆ to R₈ are as defined above;
R₉ represents a hydrogen atom, a linear C₁₋₁₀-hydrocarbon group, a hydroxy group, or a substituted or unsubstituted benzyl group; and,
the C ring has the maximum number of double bonds at the dotted line portion)

or a pharmaceutically acceptable salt thereof.

### TECHNICAL FIELD

The present invention relates to an anti-viral agent against a virus such as those belonging to the family Flaviviridae, for example.

### BACKGROUND ART

Examples of viruses belonging to the family Flaviviridae include viruses belonging to the genus Flavivirus such as yellow fever virus (YFV), dengue fever virus (DENV), Japanese encephalitis virus (JEV), and West Nile virus (WNV), viruses belonging to the genus Pestivirus such as bovine viral diarrhea virus (BVDV), and viruses belonging to the genus hepacivirus such as hepatitis C virus (HCV). Among such viruses belonging to the family Flaviviridae listed herein, viruses other than the bovine viral diarrhea virus are known to cause serious infectious diseases in humans. In particular, there are many patients throughout the world who suffer from dengue fever or hepatitis C caused by dengue fever virus or hepatitis C virus, respectively. Also recently, West Nile fever is prevalent, mainly in North America. West Nile fever is caused by the above West Nile virus.

Meanwhile, examples of known anti-viral compounds include a pyranoindole derivative (JP Patent Publication (Kohyo) No. 2005-531572 A; JP Patent Publication (Kohyo) No. 2007-526320 A; and JP Patent Publication (Kohyo) No. 2005-533031 A) to be used in treatment against hepatitis C virus, an eudistomin derivative (International Patent Publication WO2005/082373 pamphlet and International Patent Publication WO2006/088191 pamphlet) having anti-viral effects against viruses such as hepatitis C virus, a tetrazoloquinoline compound (JP Patent Publication (Kohyo) No. 2007-506788 A) to be used in an agent for inhibiting infection with hepatitis C virus, and a bicyclic imidazole derivative (JP Patent Publication (Kohyo) No. 2007-501189 A) to be used in treatment against infection with viruses of the family Flaviviridae. However, the antiviral activity of these conventional compounds is insufficient.

### DISCLOSURE OF THE INVENTION

### Object to be Attained by the Invention

In view of the above circumstances, an object of the present invention is to provide an anti-viral agent comprising a compound that exerts antiviral activity against viruses including viruses belonging to the family Flaviviridae.

### Means for Attaining the Object

As a result of intensive studies to achieve the above object, the present inventors have found that a specific heterocyclic aromatic compound has antiviral activity. Thus, the present inventors have completed the present invention.

The present invention relates to an anti-viral agent comprising a compound represented by the following formula (I):

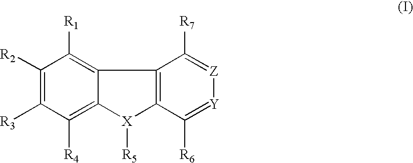

(wherein X represents CH, a nitrogen atom, an oxygen atom, or a sulfur atom; Y and Z are the same or different and each represents a nitrogen atom or C—R₈, and at least one of them represents a nitrogen atom; R₁ to R₈ are the same or different and each represents a hydrogen atom, a linear C₁₋₁₀-hydrocarbon group, a hydroxy group, or a substituted or unsubstituted benzyl group; and when X represents an oxygen atom or a sulfur atom, R₅ is absent), or the following formula (II):

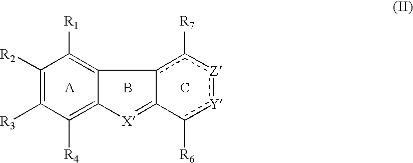

(wherein X′ represents CH or a nitrogen atom; Y′ and Z′ are the same or different and each represents a nitrogen atom or N—R₉, or C—R₈ and at least one of them represents a nitrogen atom or N—R₉; R₁ to R₄ and R₆ to R₈ are as defined above; R₉ represents a hydrogen atom, a linear C₁₋₁₀-hydrocarbon group, a hydroxy group, or a substituted or unsubstituted benzyl group; and the C ring has the maximum number of double bonds at the dotted line portion), or a pharmaceutically acceptable salt thereof.

An example of a compound represented by the above formula (I) is a compound wherein X represents a nitrogen atom; Y represents C—R₈; Z represents a nitrogen atom; R₁ to R₈ are the same or different and each represents a hydrogen atom or a linear C₁₋₁₀-hydrocarbon group.

An example of a compound represented by the above formula (II) is a compound wherein X′ represents a nitrogen atom; Y′ represents C—R₈; Z′ represents N—R₉; R₁ to R₄ and R₆ to R₉ are the same or different and each represents a hydrogen atom or a linear C₁₋₁₀-hydrocarbon group.

Also, an example of the above linear C₁₋₁₀-hydrocarbon group is a linear C₁₋₁₀-alkyl group. Moreover, an example of the above linear C₁₋₁₀-alkyl group is a methyl group.

Examples of viruses targeted by the anti-viral agent according to the present invention include viruses belonging to the family Flaviviridae.

### EFFECTS OF THE INVENTION

According to the present invention, an anti-viral agent having antiviral activity higher than that of conventional anti-viral agents can be provided.

This description includes part or all of the contents as disclosed in the descriptions of Japanese Patent Application Nos. 2007-279648 and 2008-050771, which are priority documents of the present application.

### BEST MODES FOR CARRYING OUT THE INVENTION

Hereinafter, the present invention will be described in detail. Examples of a linear C₁₋₁₀-hydrocarbon group represented by R₁ to R₉ in the above formula (I) or (II) include a linear C₁₋₁₀-alkyl group such as a methyl group, an ethyl group, a propyl group, a butyl group, a pentyl group, a hexyl group, a heptyl group, an octyl group, a nonyl group, and a decyl group; a linear C₂₋₁₀-alkenyl group such as a vinyl group, a 1-propenyl group, an allyl group, a 1-butenyl group, a 2-butenyl group, a pentenyl group, and a hexenyl group; and a linear C₂₋₁₀-alkynyl group such as an ethinyl group, a 1-propynyl group, a 2-propynyl(propargyl) group, a 3-butynyl group, a pentynyl group, and a hexynyl group.

A benzyl group represented by R₁ to R₉ in the above formula (I) or (II) may be substituted with one or more substituents selected from among a halogen atom, a heteroaromatic ring group, an acyl group, a hydroxy group, a carboxyl group, a C₁₋₁₂-hydrocarbon-O-group, and the like.

Here, examples of a halogen atom include a fluorine atom, a chlorine atom, a bromine atom, and an iodine atom.

Examples of a heteroaromatic ring group include a furyl group, a thienyl group, a pyrrolyl group, an oxazolyl group, an isoxazolyl group, a thiazolyl group, an isothiazolyl group, an imidazolyl group, a pyrazolyl group, a pyridyl group, a pyrimidinyl group, a pyridazinyl group, a pyrazinyl group, a quinolyl group, and an isoquinolyl group.

Examples of an acyl group include C₁₋₆-aliphatic acyl groups such as a formyl group, an acetyl group, a propanoyl group, a butanoyl group, a pentanoyl group, and a hexanoyl group; and aroyl groups such as a benzoyl group and a toluoyl group.

Examples of a C₁₋₁₂-hydrocarbon-O-group include C₁₋₆-alkoxy groups such as a methoxy group, an ethoxy group, a propoxy group, an isopropoxy group, a butoxy group, an isobutoxy group, a sec-butoxy group, a tert-butoxy group, a pentyloxy group, an isopentyloxy group, a hexyloxy group, a cyclopropyloxy group, a cyclobutyloxy group, a cyclopentyloxy group, and a cyclohexyloxy group.

A compound represented by the above formula (I) is preferably γ-carboline (or referred to as 5-carboline) or a derivative thereof, wherein X represents a nitrogen atom; Y represents C—R₈; and Z represents a nitrogen atom. At this time, in formula (I) above, R₁ to R₈ are the same or different and each preferably represents a hydrogen atom or a linear C₁₋₁₀-hydrocarbon group. As a linear C₁₋₁₀-hydrocarbon group, a linear C₁₋₁₀-alkyl group is particularly preferable. Further preferably, a linear C₁₋₁₀-alkyl group is a methyl group.

Also, as a compound represented by formula (II) above, a compound (corresponding to a tautomer of the above γ-carboline) or a derivative thereof is preferred, wherein X′ represents a nitrogen atom; Y′ represents C—R₈; and Z′ represents N—R₉. At this time, preferably R₁ to R₄ and R₆ to R₉ in the above formula (II) are the same or different and each represents a hydrogen atom or a linear C₁₋₁₀-hydrocarbon group. As such linear C₁₋₁₀-hydrocarbon group, a linear C₁₋₁₀-alkyl group is particularly preferred. Further preferably, a linear C₁₋₁₀-alkyl group is a methyl group.

Examples of a pharmaceutically acceptable salt of a compound represented by the above formula (I) or (II) include a salt with inorganic acid such as hydrochloric acid, sulfuric acid, phosphoric acid, hydrobromic acid, hydroiodic acid, nitric acid, pyrosulfuric acid, and metaphosphatic acid; and a salt with organic acid such as citric acid, benzoic acid, acetic acid, propionic acid, fumaric acid, maleic acid, and sulfonic acid (e.g., methanesulfonic acid, p-toluenesulfonic acid, and naphthalenesulfonic acid). Also, when the compound has a phenolic hydroxyl group or a carboxyl group, it can also be used as an alkali metal salt such as a sodium salt or a potassium salt.

A compound represented by the above formula (I), such as γ-carboline, can be produced by the method described in T. Iwaki et al., J. Chem. Soc., Perkin Trans. 1, 1999, No. 11, p. 1505-1510. β-carboline wherein X and Y each represent nitrogen; Z represents CH; and R₁ to R₇ each represent a hydrogen atom is marketed from Tokyo Chemical Industry Co., Ltd., for example. Also, a compound represented by the above formula (I) (wherein X represents a nitrogen atom; Y represents C—R₈; Z represents a nitrogen atom; one of R₁ to R₄ and R₆ to R₈ represents a methyl group; and R₁ to R₈ other than the methyl group each represent a hydrogen atom) can be produced by the method described in L. K. Dalton et al., Aust. J. Chem., 1969, Vol. 22, p. 185-195. Furthermore, a compound represented by the above formula (I) (wherein X represents a nitrogen atom; Y represents C—R₈; Z represents a nitrogen atom; R₅ represents a methyl group, and R₁ to R₄ and R₆ to R₈ each represent a hydrogen atom) can be produced by the method described in H. Zhang and R. C. Larock, J. Org. Chem., 2002, Vol. 67, p. 7048-7056.

Also, a compound represented by the above formula (I) (wherein X represents a nitrogen atom; Y represents C—R₈; Z represents a nitrogen atom; R₅ represents a methyl group; any one of R₁, R₂, R₄, and R₆ to R₈ represents a methyl group; and R₁ to R₄ and R₆ to R₈ other than the methyl group each represent a hydrogen atom) can be produced by the method described in X. Jiang et al., Org. Proc. Res. Develop., 2001, Vol. 5, p. 604-608. Furthermore, a compound represented by the above formula (I) (wherein X represents a nitrogen atom; Y represents C—R₈; Z represents a nitrogen atom; R₃ and R₅ each represent a methyl group; and R₁, R₂, R₄ and R₆ to R₈ each represent a hydrogen atom) can be produced by the method described in T. Tsunoda et al., Chem. Lett., 1994, Vol. 23, p. 539-542.

Furthermore, a compound represented by the above formula (II) (for example, wherein X′ represents a nitrogen atom; Y′ represents C—R₈; Z′ represents N—R₉; R₉ represents a methyl group; and R₁ to R₄ and R₆ to R₈ each represent a hydrogen atom) can be produced by the method described in N. N. Smolyar et al., Pharm. Chem. J., 2001, Vol. 35, p. 514-517.

Hereinafter, a compound represented by the above formula (I) or (II) and a pharmaceutically acceptable salt thereof (hereinafter, referred to as “the compound according to the present invention”) are described in terms of dosage and formulation.

The compound according to the present invention can be administered to animals and humans either directly or together with a pharmaceutical carrier commonly used. Its dosage form is not particularly limited and is appropriately selected as required for use. Examples thereof include: oral formulations such as tablets, capsules, granules, fine granules, and powders; and parenteral formulations such as injections and suppositories.

For oral formulations to exert their effects as intended, the dose (weight) of the compound according to the present invention ranges from 5 to 1,000 mg and preferably ranges from 10 to 600 mg, which is generally administered to an adult once a day or in several separated doses, but differs depending on age, body weight, and the degree of disease of a patient.

Such oral formulations are produced according to a conventional method using starch, lactose, saccharose, mannite, carboxymethyl cellulose, corn starch, or inorganic salts, for example.

For such kinds of formulations, in addition to the above appropriate excipients, a binder, a disintegrator, a surfactant, a lubricant, an agent for accelerating flowability, a flavoring agent, a colorant, an aroma chemical, and the like can be used.

Examples of a binder include starch, dextrin, gum Arabic powder, gelatin, hydroxypropyl starch, methylcellulose, sodium carboxymethylcellulose, hydroxypropylcellulose, crystalline cellulose, ethyl cellulose, polyvinylpyrrolidone, and Macrogol.

Examples of a disintegrator include starch, hydroxypropyl starch, sodium carboxymethylcellulose, carboxymethylcellulose calcium, carboxymethylcellulose, and low substituted hydroxypropyl cellulose.

Examples of a surfactant include sodium lauryl sulfate, soybean lecithin, sucrose fatty acid ester, and polysorbate 80.

Examples of a lubricant include talc, waxes, hydrogenated plant oil, sucrose fatty acid ester, magnesium stearate, calcium stearate, aluminum stearate, and polyethylene glycol.

Examples of an agent for accelerating flowability include light anhydrous silicic acid, dried aluminum hydroxide gel, synthetic aluminum silicate, and magnesium silicate.

Also, the compound according to the present invention can be administered in the form of suspension, emulsion, syrup, or elixir. Various dosage forms thereof may contain a taste and flavor corrigent or a colorant.

For a parenteral formulation to exert its effects as predetermined, the dose (weight) of the compound according to the present invention generally ranges from 5 to 500 mg per day and preferably 10 to 300 mg per day, which is adequately administered to an adult via intravenous injection, IV infusion, subcutaneous injection, or intramuscular injection, but differs depending on age, body weight, and the degree of disease of a patient.

Such parenteral formulation is produced according to a conventional method. Distilled water for injection, a saline, a glucose aqueous solution, olive oil, sesame oil, peanut oil, soybean oil, corn oil, propylene glycol, polyethylene glycol, or the like can generally be used as a diluent. If necessary, a germicide, antiseptic, stabilizer, or the like may further be added thereto. Moreover, in light of stability, a vial or the like is charged with such parenteral formulation and then frozen, followed by removal of water by a general freeze-drying technique, and a liquid formulation can be prepared again from the freeze-dried product immediately before use. If necessary, tonicity agents, stabilizers, antiseptics, soothing agents, and so on may be added appropriately.

Other examples of such parenteral formulation include adhesive skin patches, liquid formulations for external use, liniments such as paste, and suppositories for intrarectal administration, which are produced by conventional methods.

Meanwhile, the compound according to the present invention can be used for inhibiting viral infection of viruses. Examples of such viruses include, but are not particularly limited to, viruses belonging to the family Flaviviridae, the family Togaviridae, the family Reoviridae, the family Picornaviridae, the family Bunyaviridae, the family Orthomyxoviridae, the family Paramyxoviridae, the family Coronaviridae, the family Caliciviridae, the family Adenoviridae, the family Papovaviridae, the family Poxyiridae, the family Rhabdoviridae, the family Herpesviridae, the family Arenaviridae, or the family Retroviridae. Particularly, the compound according to the present invention can be used for inhibiting infection with viruses belonging to the family Flaviviridae. Examples of viruses belonging to the family Flaviviridae include viruses belonging to the genus Flavivirus such as yellow fever virus (YFV), dengue fever virus (DENV), Japanese encephalitis virus (JEV), and West Nile virus (WNV); viruses belonging to the genus Pestivirus such as bovine viral diarrhea virus (BVDV); and viruses belonging to the genus hepacivirus such as hepatitis C virus (HCV).

The antiviral activity of the compound according to the present invention can be evaluated by a method that involves infecting cells with a virus, adding the compound according to the present invention to medium before, after, or simultaneously with infection, and then measuring the percent inhibition of viral replication. Specifically, such percent inhibition of viral replication can be evaluated by measuring the activity of lactate dehydrogenase (LDH) in the culture supernatant of the virus-infected cells using an LDH cytotoxicity detection kit (Takara Biochemicals), for example. LDH is an enzyme that exists in the cytoplasm and is generally almost never released extracellularly because of the presence of cell membrane. Meanwhile, when cells are infected with a virus and then the virus replicated within the cells, infected cells die. As a result of viral infection, cell membrane is disrupted and then LDH is released into the culture supernatant. When virus-infected cells are cultured in the presence of the compound according to the present invention, viral infection or replication is inhibited by the effects of the compound according to the present invention. However, the membranes of the cells are not disrupted, so that an LDH level in the culture supernatant shows no increase. That is, the degree of cell disruption due to viral replication and the LDH level in the culture supernatant show extremely good positive correlation. Therefore, the antiviral activity (inhibition of viral replication) of the compound according to the present invention can be measured via quantitative determination of LDH in the culture supernatant.

Also, after addition of the compound according to the present invention to medium containing cells not infected with any virus, the percent inhibition of cell growth of the compound according to the present invention is measured. For example, with the use of a reagent for viable cell measurement, such as a water soluble MTT solution TetraColor One™ (Seikagaku Corporation), the percent inhibition of cell growth can be measured.

Next, the concentration (that is, 50% effective concentration: EC₅₀) of the compound according to the present invention, which yields 50% inhibition of viral replication, is calculated from the thus obtained percent inhibition of viral replication. Meanwhile, the concentration (that is, 50% cytotoxicity concentration: CC₅₀) of the compound according to the present invention, which yields 50% inhibition of cell growth, is calculated from the thus obtained percent inhibition of cell growth. Furthermore, the selectivity index (CC₅₀/EC₅₀) is calculated. The higher the selectivity index, the higher the effects of inhibiting viral replication alone without damaging cells. Therefore, the antiviral activity of the compound according to the present invention can be evaluated using the selectivity index as an index. Also, the antiviral activity of the compound according to the present invention can be evaluated by assay (e.g., Western blot analysis, ELISA, or flow cytometry) for measuring the viral antigen level in virus-infected cells cultured in medium containing the compound according to the present invention or assay (e.g., Northern blot analysis or quantitative RT-PCR) for measuring the virus gene (RNA) level in virus-infected cells.

As described above, viral replication can be inhibited by administering an anti-viral agent containing the compound according to the present invention as an active ingredient to a virus-infected subject.

### EXAMPLES

Hereafter, the present invention is described in detail with reference to Examples, although the technical scope of the present invention is not limited thereto.

### Reference Example

### Antiviral Activity of Fluorene, Dibenzofuran, and Carbazole

### (1) Materials

The following materials were used for examining the antiviral activity of fluorene, dibenzofuran, and carbazole:

1) Cell: Madin-Darby bovine kidney cell (hereinafter, referred to as “MDBK cell”);

2) Virus: Bovine viral diarrhea virus (hereinafter, referred to as “BVDV”) Nose strain;

3) Medium: Dulbecco's Modified Eagle Medium supplemented with 100 units/ml penicillin G, 100 μg/ml streptomycin, and 3% horse serum;

4) Culture plate: 96-well flat bottom microtiter plate;

5) Reagent or kit for measurement: LDH cytotoxicity detection kit (Takara Biochemicals) and water soluble MTT solution TetraColor One™ (Seikagaku Corporation);

6) Compound:

Compounds tested are as listed in Table 1 below.

TABLE 1

| Compound | Chemical formula |
|---|---|
| Ribavirin | — |
| (1-β-D-ribofuranousyl- | |
| 1,2,4-triazole-3-carboxamide | |
| Cyclosporin A | — |
| Interferon-α | — |
| Fluorene | 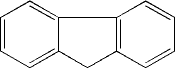 |
| Dibenzofuran | 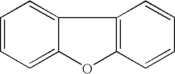 |
| Carbazole | 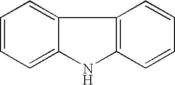 |

Ribavirin manufactured by Schering-Plough was used. Cyclosporin A manufactured by Sigma was used. Furthermore, interferon-α manufactured by PBL Biochemical Laboratories was used.

Furthermore, fluorine manufactured by Aldrich was used. Also, dibenzofuran and carbazole manufactured by Kanto Chemical Co., Inc. were used.

Among the above compounds, Ribavirin is a nucleic acid derivative known as having anti-BVDV effects and anti-hepatitis C virus (hereinafter, referred to as “HCV”) effects. Ribavirin is currently clinically used as a therapeutic agent together with an interferon against hepatitis C. Also, the anti-HCV effects of cyclosporin A have been demonstrated in vitro. Moreover, interferon-α has anti-viral effects against a wide range of viruses. Specifically, its anti-BVDV effects and anti-HCV effects have been demonstrated. In this Reference Example, Ribavirin, cyclosporin A, and interferon-α were used as positive controls.

Also, the above LDH cytotoxicity detection kit is a kit for measuring cell damage through measurement of lactate dehydrogenase (LDH) released from cells. In this Reference Example, the kit was used for calculating the percent inhibition of viral replication. Furthermore, the water soluble MTT solution TetraColor One™ is a reagent for measuring viable cells.

### (2) Method

The antiviral activity of Ribavirin, cyclosporin A, and interferon-α, as well as fluorene, dibenzofuran, and carbazole, were measured by the following method (Baba C. et al., Antiviral Chem. Chemother., 16: 33-39 (2005)).

MDBK cells (2×10⁵ cells/ml) were infected with BVDV at an MOI of 0.01 (multiplicity of infection, MOI=0.01). Next, a solution containing BVDV-infected cells was dispensed into a 96-well flat bottom microtiter plate at 100 μl per well, simultaneously with the addition of a compound subjected to 5-fold serial dilution, followed by 3 days of culture at 37° C. (5% CO₂).

After 3 days of culture, 50 μl of the cultured supernatant was collected and then transferred to another microtiter plate and then 50 μl of the reaction solution of an LDH cytotoxicity detection kit was added. After 30 minutes of culture at room temperature, the microtiter plate was applied to a microplate reader (BioRad Laboratories) and then absorbance was measured at 490 nm/690 nm.

The percent inhibition (%) of viral replication was calculated by the following formula based on the thus obtained absorbance:

100−[(OD_{T})_{V}−(OD_{C})_{M}]/[(OD_{C})_{V}−(OD_{C})_{M}]×100(%)

In this formula, each abbreviation represents the following:

(OD_{T})_{V}: Absorbance (LDH activity) of a culture supernatant of the virus-infected cells in the presence of the compound;

(OD_{C})_{M}: Absorbance (LDH activity) of a culture supernatant of the uninfected cells in the absence of the compound;

(OD_{C})_{V}: Absorbance (LDH activity) of a culture supernatant of the virus-infected cells in the absence of the compound.

Furthermore, the concentration (50% effective concentration: EC₅₀) of a compound, which yielded 50% inhibition of viral replication, was calculated from the thus obtained percent inhibition of viral replication.

At the same time, for measurement of toxicity of each compound, the compound was added to a microtiter plate containing MDBK cells not infected with the virus as described above, followed by 3 days of culture. After 3 days of culture, TetraColor One™ was added to a microtiter plate at 10 μl per well. After 1 hour of incubation at 37° C., the microtiter plate was applied to a microplate reader and then absorbance was measured at 450 nm/690 nm.

The percent inhibition (%) of cell growth was calculated by the following formula based on the thus obtained absorbance:

100−[(OD_{T})_{M}/(OD_{C})_{M}]×100(%)

In this formula, each abbreviation represents the following:

(OD_{T})_{M}: Absorbance (MTT activity) of a culture medium of the uninfected cells in the presence of a compound;

(OD_{C})_{M}: Absorbance (MTT activity) of a culture medium of the uninfected cells in the absence of a compound.

The concentration (50% cytotoxicity concentration CC₅₀) of a compound, which yielded 50% inhibition of cell growth, was calculated from the thus obtained percent inhibition of cell growth.

### (3) Results

Table 2 shows the EC₅₀(μM), CC₅₀(μM), and selectivity index (CC₅₀/EC₅₀) of each compound. In addition, values of EC₅₀(μM) and CC₅₀(μM) are each mean value calculated from the values obtained by an experiment that was conducted separately at least twice.

TABLE 2

| | | | Selectivity index |
|---|---|---|---|
| Compound | EC₅₀(μM) | CC₅₀(μM) | (CC₅₀/EC₅₀) |
| Ribavirin | 3.9 | 15.1 | 3.9 |
| Cyclosporin A | 2.8 | 16.1 | 5.8 |
| Interferon-α | 5.5* | >100* | >18.2 |
| Fluorene | >100 | >100 | <>1 |
| Dibenzofuran | >100 | >100 | <>1 |
| Carbazole | >100 | >100 | <>1 |

*The unit for EC₅₀ and CC₅₀ of interferon-α is International unit/well (IU/well).

As is understood from Table 2, existing drugs, Ribavirin and cyclosporin A exerted selective anti-BVDV effects, but their selectivity indexes were each 10 or less, which was not so high.

### Example 1

### Antiviral Activity of β-Carboline and γ-Carbo Line

According to the method described in the Reference Example above, the antiviral activity of β-carboline and that of γ-carboline were measured, as shown in Table 3 below. β-carboline manufactured by Tokyo Chemical Industry Co., Ltd. was used. γ-carboline was produced according to the method described in T. Iwaki et al., J. Chem. Soc., Perkin Trans. 1, 1999, No. 11, p. 1505-1510.

Also, Table 3 shows the EC₅₀(μM), CC₅₀(μM), and selectivity index (CC₅₀/EC₅₀) of β-carboline and γ-carboline as measured.

TABLE 3

| | | | | Selectivity |
|---|---|---|---|---|
| | | | | index |
| | | EC₅₀ | CC₅₀ | (CC₅₀/ |
| Compound | Chemical formula | (μM) | (μM) | EC₅₀) |
| β-Carboline | 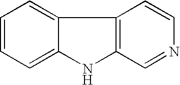 | 8.5 | 87 | 10.2 |
| γ-Carboline | 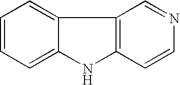 | 2.1 | 41 | 19.5 |

As is understood from Table 3, β-carboline and γ-carboline exerted selective anti-BVDV effects. Particularly γ-carboline had strong effects.

### Comparative Example

According to the above methods described in Reference Example above, the antiviral activity of α-carboline and that of 6-carboline were measured as shown in Table 4 below. These types of carboline were produced according to the method described in T. Iwaki et al., J. Chem. Soc., Perkin Trans. 1, 1999, No. 11, p. 1505-1510.

Also, Table 4 shows the EC₅₀(μM), CC₅₀(μM), and selectivity index (CC₅₀/EC₅₀) of α-carboline and δ-carboline as measured.

TABLE 4

| | | | | Selectivity |
|---|---|---|---|---|
| | | | | index |
| | | EC₅₀ | CC₅₀ | (CC₅₀/ |
| Compound | Chemical formula | (μM) | (μM) | EC₅₀) |
| α-Carboline | 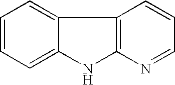 | 91 | >100 | >1.1 |
| δ-Carboline | 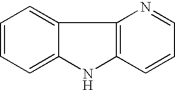 | >100 | >100 | <>1 |

As is understood from Table 4, the anti-BVDV effects of α-carboline and δ-carboline were lower than those of β-carboline and γ-carboline (Table 3).

### Example 2

### Antiviral Activity (1) of γ-Carboline Derivative

According to the method described in the Reference Example above, the antiviral activity of methyl-γ-carboline was measured as shown in Table 5 below. Compounds 1, 3, 4 and 6 to 9 shown in Table 5 were produced according to the method described in L. K. Dalton et al., Aust. J. Chem., 1969, Vol. 22, p. 185-195. Also, compound 5 shown in Table 5 was produced according to the method described in H. Zhang and R. C. Larock, J. Org. Chem., 2002, Vol. 67, p. 7048-7056. Furthermore, compound 2 shown in Table 5 was produced according to the method described in N. N. Smolyar et al., Pharm. Chem. J., 2001, Vol. 35, p. 514-517.

Also, Table 5 shows the EC₅₀(μM), CC₅₀(μM), and selectivity index (CC₅₀/EC₅₀) of each methyl-γ-carboline as measured.

TABLE 5

| | | | | Selectivity |
|---|---|---|---|---|
| Methyl-γ-carboline | | | | index |
| (Compound No.) | Chemical formula | EC₅₀ (μM) | CC₅₀ (μM) | (CC₅₀/EC₅₀) |
| 1 | 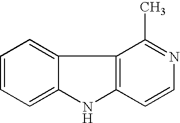 | 0.58 | 9.7 | 16.7 |
| 2 | 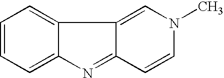 | 4.3 | 65 | 15.1 |
| 3 | 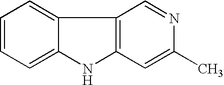 | 2.2 | 70 | 31.8 |
| 4 | 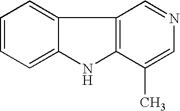 | 0.55 | 21 | 38.2 |
| 5 | 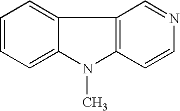 | 0.26 | 29 | 111.5 |
| 6 | 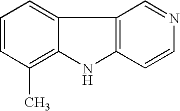 | 1.7 | 24 | 14.1 |
| 7 | 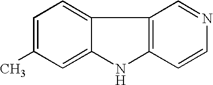 | 1.1 | 24 | 21.8 |
| 8 | 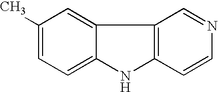 | 3.0 | 12 | 4 |
| 9 | 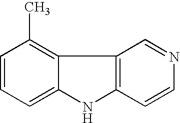 | 1.2 | 16 | 13.3 |

As is understood from Table 5, all 9 types of methyl-γ-carboline were found to have selective anti-BVDV effects. In particular, compound 5 was found to have the strongest activity and the highest selectivity.

### Example 3

### Antiviral Activity (2) of γ-Carboline Derivative

According to the method described in the Reference Example above, the antiviral activity of dimethyl-γ-carboline was measured as shown in Table 6 below. Compounds 10 to 13, 15 and 16 shown in Table 6 were produced according to the method described in X. Jiang et al., Org. Proc. Res. Develop., 2001, Vol. 5, p. 604-608. Also, compound 14 shown in Table 6 was produced according to the method described in T. Tsunoda et al., Chem. Lett., 1994, Vol. 23, p. 539-542.

Also, Table 6 shows the EC₅₀(μM), CC₅₀(μM), and selectivity index (CC₅₀/EC₅₀) of each dimethyl-γ-carboline as measured.

TABLE 6

| Dimethyl-γ-carboline | | | | Selectivity index |
|---|---|---|---|---|
| (Compound No.) | Chemical formula | EC₅₀ (μM) | CC₅₀ (μM) | (CC₅₀/EC₅₀) |
| 10 | 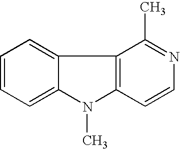 | 0.20 | 5.2 | 26.0 |
| 11 | 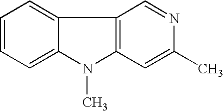 | 0.062 | 21 | 338.7 |
| 12 | 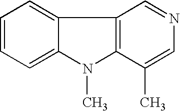 | 0.043 | 9.6 | 223.2 |
| 13 | 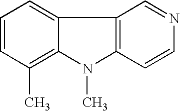 | 0.30 | 11 | 36.7 |
| 14 | 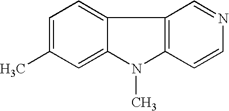 | 0.30 | 40 | 133.3 |
| 15 | 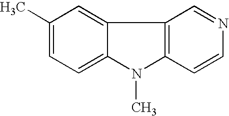 | 0.35 | 7.7 | 22.0 |
| 16 | 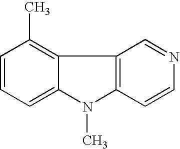 | 0.29 | 6.4 | 22.1 |

As is understood from Table 6, all 7 types of dimethyl-γ-carboline were found to have selective anti-BVDV effects. In particular, compound 12 was found to have the strongest activity and high selectivity. Also, compound 11 was found to have activity equivalent to that of compound 12 and the highest selectivity.

It is easily inferred that, among viruses belonging to the family Flaviviridae the structures of the molecule targeted by the compound according to the present invention are the same as or extremely analogous to each other. Therefore, it can be expected that the compound according to the present invention also exerts strong antiviral activity against other viruses belonging to the family Flaviviridae, as in the case of BVDV.

All publications, patents, and patent applications cited herein are incorporated herein by reference in their entirety.

1. An anti-viral agent comprising (A) a compound represented by the following formula (I): 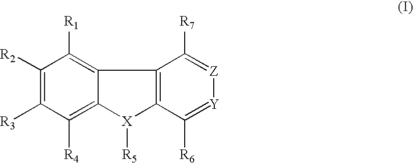
wherein X represents CH, a nitrogen atom, an oxygen atom or a sulfur atom;
Y and Z are the same or different and each represents a nitrogen atom or C—R₈, and at least one of them represents a nitrogen atom;
R₁ to R₈ are the same or different and each represents a hydrogen atom, a linear C₁₋₁₀-hydrocarbon group, a hydroxy group, or a substituted or unsubstituted benzyl group; and,
when X represents an oxygen atom or a sulfur atom, R₅ is absent, or by the following formula (II): 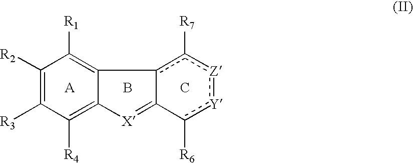
wherein X′ represents CH or a nitrogen atom;
Y′ and Z′ are the same or different and each represents a nitrogen atom or N—R₉, or C—R₈ and at least one of them represents a nitrogen atom or N—R₉;
R₁ to R₄ and R₆ to R₈ are as defined above;
R₉ represents a hydrogen atom, a linear C₁₋₁₀-hydrocarbon group, a hydroxy group, or a substituted or unsubstituted benzyl group and,
the C ring has the maximum number of double bonds at the dotted line portion,
or (B) a pharmaceutically acceptable salt thereof.

2. The anti-viral agent according to claim 1, comprising the compound or a pharmaceutically acceptable salt thereof, wherein, in formula (I), X represents a nitrogen atom, Y represents C—R₈, Z represents a nitrogen atom, and R₁ to R₈ are the same or different and each represents a hydrogen atom or a linear C₁₋₁₀-hydrocarbon group.

3. The anti-viral agent according to claim 1, comprising the compound or a pharmaceutically acceptable salt thereof, wherein, in formula (II), X′ represents a nitrogen atom, Y′ represents C—R₈, Z′ represents N—R₉, and R₁ to R₄ and R₆ to R₉ are the same or different and each represents a hydrogen atom or a linear C₁₋₁₀-hydrocarbon group.

4. The anti-viral agent according to claim 1, wherein the linear C₁₋₁₀-hydrocarbon group is a linear C₁₋₁₀-alkyl group.

5. The anti-viral agent according to claim 4, wherein the linear C₁₋₁₀-alkyl group is a methyl group.

6. The anti-viral agent according to claim 1, wherein the virus belongs to the family Flaviviridae.
